(19) United States
(12) Patent Application Publication
(10) Pub. No.: US 2016/0271073 A1
(43) Pub. Date: Sep. 22, 2016

(54) THERAPEUTIC SCREEN PRINTING INK FOR APPAREL

(71) Applicant: Robert Falken, Oceanside, CA (US)

(72) Inventor: Robert Falken, Oceanside, CA (US)

(21) Appl. No.: 15/072,297

(22) Filed: Mar. 16, 2016

Related U.S. Application Data
(60) Provisional application No. 62/133,893, filed on Mar. 16, 2015.

(51) Int. Cl. A61K 9/70; A41D 27/08; C09D 11/03; A61K 33/22; A61K 47/32

(52) U.S. Cl. CPC A61K 9/70; A61K 33/22; A61K 47/32; C09D 11/03; A41D 27/08; A41D 2400/32

(57) ABSTRACT

An article, article of manufacture, and method of making a therapeutic apparel item are disclosed. An article includes a piece of clothing formed of a fabric and adapted to be worn on a wearer's person. The article further includes ink applied to a portion of a surface of the piece of clothing, the ink comprising a blend of screen printing ink and negative ion powder.

### CROSS-REFERENCE TO RELATED APPLICATION

This application claims priority to and the benefit of U.S. Provisional Patent Application No. 62/133,893, filed on Mar. 16, 2015 and titled “THERAPEUTIC SCREEN PRINTING INK FOR APPAREL,” the disclosure of which is incorporated by reference in its entirety.

### BACKGROUND

Of late, considerable interest has arisen in the claimed therapeutic effects of negative ions, particularly for improving athletic performance and increased blood circulation in a person. Recently published reports indicate that there well may be some merit to these claims.

Negative ions are odorless, tasteless, and invisible molecules that are inhaled or absorbed by a person in certain environments. Examples of environments rich in negative ions are areas containing waterfalls, crashing waves on beaches, or clean mountain air. Once negative ions reach a person's bloodstream, they are believed to produce biochemical reactions that increase levels of the mood enhancing chemical serotonin, helping to alleviate depression, relieve stress, and improve a person's sense of well-being and happiness. The understanding that human beings are bioelectric in nature, and electrically charged particles affect the way a person feels and acts supports this.

Several thousand scientific documents based on research in Israel, Europe, and in Asia support the concept that high doses of negative ions emit positive effects on a person, while the opposite is true when a person is exposed to high amounts of positive ions. Recent research has shown that negative ions may also protect against germs in the air, resulting in decreased irritation due to inhaling various particles that make a person sneeze, cough, or have a throat irritation.

Several consumer products have appeared on the market within recent years, which are offered as an emitting source of negative ions. Examples of such products are silicone wrist bracelets or necklaces containing a pendant that emit negative ions. These products are not very effective due to their small surface areas. Moreover, a wearer of the present invention is more likely to inhale and absorb large quantities of negative ions since the therapeutic screen printing ink can be applied to larger surface areas such as across the chest of a t-shirt.

SUMMARY

A therapeutic screen printing ink for apparel that improves the sense of well-being and happiness of a wearer and a method of using the therapeutic screen printing ink is disclosed. The therapeutic screen printing ink contains an anion negative ion material that is added in the manufacturing process, which can release negative ions around the wearer's body. In some implementations, a therapy method includes applying a negative ion-bearing screen printing ink to a garment or accessory, and providing the printed garment or accessory to a wearer for wearing on the wearer's body for a sufficient period of time, thereby stimulating the wearer to achieve an improvement in their sense of well-being and happiness.

The details of one or more embodiments are set forth in the accompanying drawings and the description below. Other features and advantages will be apparent from the description and drawings, and from the claims.

### BRIEF DESCRIPTION OF THE DRAWINGS

These and other aspects will now be described in detail with reference to the following drawings.

FIG. 1 illustrates apparel in the form of a t-shirt with therapeutic screen printing ink applied to its surface.

FIG. 2 is a flowchart to illustrate a method of producing a therapeutic screen printing ink and applying it to apparel.

Like reference symbols in the various drawings indicate like elements.

### DETAILED DESCRIPTION

This document describes therapeutic screen printing ink. In some implementations, negative ions are embedded into the ink formulation that is applied to a garment or accessory, such as a t-shirt, shirt, pants, yoga wear, performance apparel such as undergarments (shirts and shorts or leggings), beanies, hats, earmuffs, scarves, sweatbands, or the like. Moreover, a wearer of such garment or accessory is more likely to inhale and absorb large quantities of negative ions since the therapeutic screen printing ink can be applied to larger surface areas such as across the chest of a t-shirt.

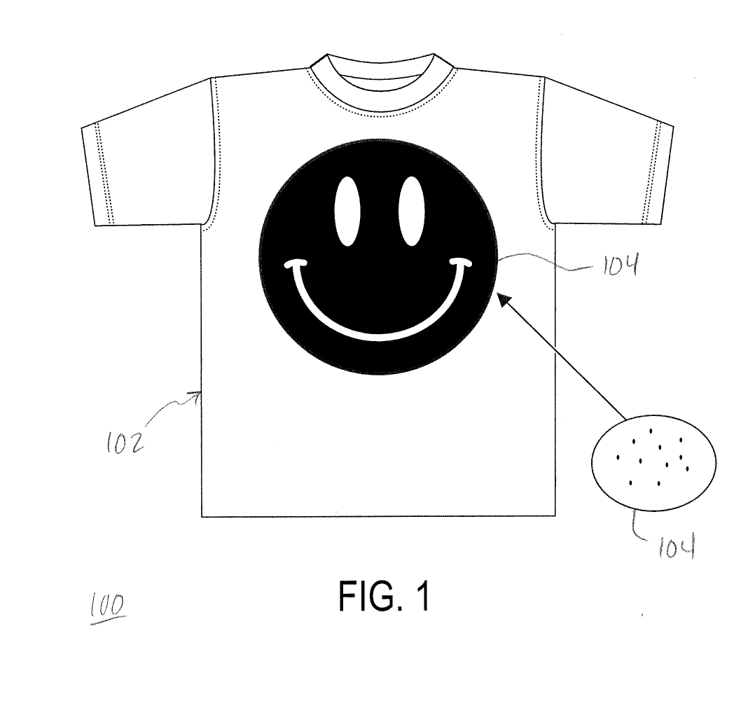

FIG. 1 shows an article 100, preferably including an article of clothing 102 for being worn by a wearer on the wearer's person. In some implementations, the article 100 is adapted to be worn in close proximity to the wearer's skin. The article of clothing 102 can be made of a fabric, which can be knitted or weaved. Alternatively, the article of clothing 102 can be made of a solid material such as leather, rubber, or the like. The article of clothing 102 includes ink 104 applied to a portion of a surface of the article of clothing 102. The ink 104 can be deposited or printed on the article of clothing 102 in any format, pattern, or shape. The ink 104 includes a blend of screen printing ink and negative ion powder 104.

In some implementations, a negative ion composition is used for the basis of the ideal negative ion-containing screen printing ink formulation. An illustrative and non-limiting example includes the use of tourmaline powder that is a crushed or pulverized form of tourmaline, a semi-precious natural gemstone mineral with a high natural output of negative ions. The preferred ink formulation of this implementation may contain any ratio of 1% to 30% tourmaline powder, or more, by weight of the, to ensure that the emitting negative ions of the negative ion composition release at a concentration between 100 and 2000, or more, negative ions per cubic centimeter. The tourmaline powder may be included directly into the ink formula at the point of manufacture.

In some implementations, a blend of negative ion powders can be utilized in the formulation. The negative ion powder may consist of a blend of germanium, rare earth, silicon oxide, alumina, rare earth, and tourmaline. Additionally, any type of negative ion emitting substance may be utilized. Additional examples of this are bio-ceramic materials such as silica, aluminum, magnesium, or rare earth magnets, which can be used in various forms.

In an alternate implementation, the negative ion portion of the therapeutic screen printing ink may be screened onto a pre-applied layer of screen printing ink that has been applied onto a garment. An overprint layer being applied to the pre-applied layer of screen printing ink achieves this with a negative ion agent disposed in the clear overcoat layer.

In yet another implementation, the negative ion portion of the formulation may be supplied in a foil transfer that is heat pressed into a clear adhesive which has been pre-screened onto a garment.

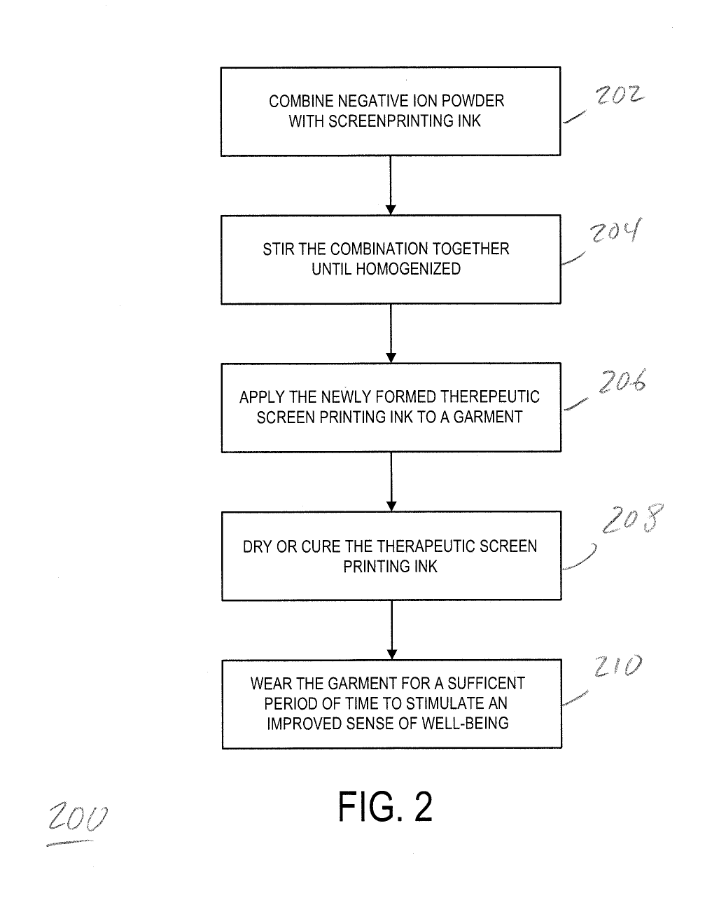

FIG. 2 is a flowchart illustrating a method 200 of producing a therapeutic screen printing ink and applying it to apparel. At 202, negative ion powder is combined with screen printing ink. At 204, the combination of negative ion powder and screen printing ink is stirred together until the combination reaches a homogenized state, i.e. the combination is able to be applied uniformly to the apparel, to form a therapeutic screen printing ink.

At 206, the therapeutic screen printing ink is applied to the apparel, which can be a garment, a piece of cloth, an accessory, or other item that can be worn by a wearer, or applied against a wearer's skin. For instance, in some implementations, the apparel can include a tape that includes an adhesive layer for being adhered to a wearer's skin, and in which the tape includes a fabric layer that includes the therapeutic screen printing ink. At 208, the applied therapeutic screen printing ink is dried or cured while on the apparel. At 210, a wearer of the apparel wears the apparel for a sufficient period of time to stimulate an improved sense of well-being, as provided and conveyed by the negative ion content of the therapeutic screen printing ink applied to the garment.

The therapeutic screen printing ink may be manufactured of one or more types of screen printing inks. In particular, inks such as water-based inks are preferred due to their deep color penetration into garments and soft hand feel. Water-based inks utilize either dyes or pigments in a suspension with water as the solvent in their creation. The evaporation of the water is necessary to set or cure the ink. This curing can take place either at room temperature or with the assist of a dryer depending upon the specific water-based ink used and the speed or volume of production. For dark colored garments the preferred water-base ink is called discharge ink. This ink can be printed as direct discharge, dye discharge, or used as an under base for additional printing. Discharge ink is type of water-based ink with the clear distinction of having a discharge agent mixed into the ink that removes manufacturer dye from the garment. Once a manufacturer's dye has been removed, the discharge ink will then re-dye that print area a desired ink color.

Yet another type of ink that is ideal for use is referred to as plastisol ink. This type of ink is comprised of either acrylic or polyvinyl chloride resin in a liquid plasticizer. Plastisol ink is a thermoplastic in that it is necessary to heat the printed ink film to a temperature high enough to cause the molecules of resin and plasticizer to crosslink and thereby solidify, or cure. The more common temperature range that most plastisol inks cure is between 300° F. to 330° F.

Although a few embodiments have been described in detail above, other modifications are possible. Other embodiments may be within the scope of the following claims.

1. An article comprising:
a piece of clothing formed of a fabric; and
ink applied to a portion of a surface of the piece of clothing, the ink comprising a blend of screen printing ink and negative ion powder.

2. An article in accordance with claim 1, wherein the ink further comprises acrylic resin in a liquid plasticizer.

3. An article in accordance with claim 1, wherein the ink further comprises polyvinyl chloride resin in a liquid plasticizer.

4. An article in accordance with claim 1, wherein the negative ion powder is comprised of tourmaline.

5. An article in accordance with claim 1, wherein the negative ion powder is selected from one or more of the negative ion powders that consist of: germanium, rare earth, silicon oxide, alumina, rare earth, tourmaline, silica, aluminum, magnesium, and/or rare earth magnets.

6. An article of manufacture comprising:
a garment for being worn on a wearer's person; and
a screen print on a portion of the garment, the screen print comprising one or more inks combined with one or more negative ion-emitting substances.

7. An article of manufacture in accordance with claim 6, wherein the ink further comprises acrylic resin in a liquid plasticizer.

8. An article of manufacture in accordance with claim 6, wherein the ink further comprises polyvinyl chloride resin in a liquid plasticizer.

9. An article of manufacture in accordance with claim 6, wherein the negative ion powder is comprised of tourmaline.

10. An article of manufacture in accordance with claim 6, wherein the negative ion powder is selected from one or more of the negative ion powders that consist of:
germanium, rare earth, silicon oxide, alumina, rare earth, tourmaline, silica, aluminum, magnesium, and/or rare earth magnets.
